(19) United States
(12) Patent Application Publication
(10) Pub. No.: US 2025/0099359 A1
(43) Pub. Date: Mar. 27, 2025

(54) METHODS FOR REDUCING FRIZZ AND IMPROVING SMOOTHNESS OF HAIR

(71) Applicant: L'OREAL, Paris (FR)

(72) Inventor: XianZhi ZHOU, Millburn, NJ (US)

(21) Appl. No.: 18/355,447

(22) Filed: Jul. 20, 2023

(51) Int. Cl. A61K 8/49; A61Q 5/06

(52) U.S. Cl. CPC A61K 8/4973; A61Q 5/06

(57) ABSTRACT

The instant disclosure is drawn to methods for reducing frizz and increasing smoothness of hair. The methods involve applying a hair treatment composition to hair in need of reduced frizz or increased smoothness, the hair treatment composition comprising: (a) propylene carbonate; and (b) water, wherein the composition is free from alkylene carbonates, cyclic carbonates other than the propylene carbonate, and N-alkyl-2-mercaptoacetamide. After rinsing the hair treatment composition from the hair, the hair is subjected to a heat treatment at a temperature of at least 150° C. The methods result in in hair having improving hair fiber alignment, reduced frizz, and smoothness.

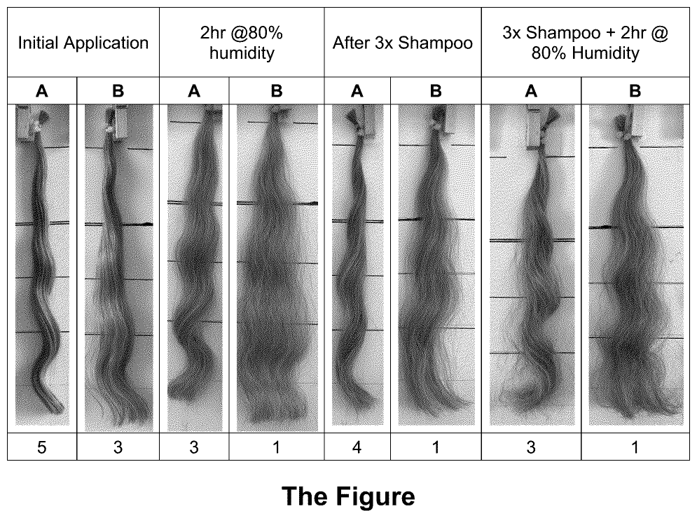

### FIELD OF THE DISCLOSURE

The instant disclosure is drawn to methods for reducing frizz and increasing smoothness of hair in need of reduced frizz or increased smoothness. Hair is treated with a rinse off hair treatment composition and subsequently treated with a thermal heat treatment. Treatment with the compositions provide hair with improved fiber alignment, frizz control, and smoothness.

### BACKGROUND

Many consumers use cosmetic and care compositions to enhance the appearance of hair, e.g., by changing the color, style, or shape of the hair and/or by imparting various cosmetic properties to hair, such as shine and conditioning. Hair can become dry or damaged for various reasons, e.g., weather exposure, poor nutrition, mechanical treatments (e.g., brushing hair), styling treatments using chemicals, dying, heat, nutrition, etc. Even cleansing products can remove hair's natural oils causing dryness, which can lead to a dull appearance, split ends, and frizz.

Chemical treatments for hair include bleaching and coloring treatments to change the color the hair. Chemical treatments also include processes to permanently change the shape and structure of the hair, for example by perming, waving, relaxing or straightening the hair. These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts.

The popularity and usage of oils for hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g., 8 hours) of treatment and several treatments are usually required, making it time consuming and labor intensive.

Damage to hair results in split ends, dryness, hair that is easily broken, and hair that becomes “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair treatment technologies that can straighten, relax, or style the hair without chemically damaging the hair are desired.

There is still a need for providing improved manageability of hair, for example, improved hair alignment, reduced unwanted volume (especially reduced frizz), and increased shine.

### SUMMARY OF THE DISCLOSURE

The instant disclosure is drawn to methods for improving the look, feel, and style of hair. In particular, the methods improve fiber alignment, reduce frizz, and impart smoothness to the hair. The treated hair is soft, shiny, conditioned, and has a healthy appearance. The methods use a hair treatment composition containing propylene carbonate, which undergoes a reaction when subjected to a thermal heat treatment.

For example, heat imparted by a hot iron activates a crosslinking or other modification reaction with amine groups of the hair fibers. Without wishing to be bound by any particular theory, the inventors believe N, N′-disubstituted urea linkages are formed with the keratin fibers of the hair. This results in a fixation of the newly formed shape. The methods are gentle and do not damage the hair. Furthermore, the improved cosmetic properties imparted to the hair are long-lasting and maintained even after multiple washing cycles.

The methods of the instant disclosure are generally drawn to reducing frizz or increasing smoothness of hair by:
(i) applying a hair treatment composition to hair in need of reduced frizz or increased smoothness; the composition comprising:
   (a) about 1 to about 15 wt. % of propylene carbonate; and
   (b) water,
      wherein the composition is free from alkylene carbonates, cyclic carbonates other than the propylene carbonate, and N-alkyl-2-mercaptoacetamide, and
      all weight percentages are based on a total weight of the composition;
(ii) after without rinsing the composition from the hair, subjecting the hair to a heat treatment, wherein the heat treatment heats the hair to a temperature of at least 150° C.

In various embodiments, the hair treatment composition has an acidic pH, for example, a pH less than 7, or a pH of from about 3 to about 6.

The hair treatment composition can be applied to dry hair or to wet/damp hair. The hair treatment compositions contain water. Therefore, application of the hair treatment composition to the hair will impart a degree of wetness to the hair. Accordingly, in various embodiments, after application of the hair treatment composition, the hair is dried after rinsing the hair treatment composition from the hair, for example, using a blow dryer. Most blow dryers reach a maximum temperature of about 90° C. to about 120° C. Therefore, after the hair is dried, the hair may be treated with a hot iron (preferably a flat iron) or other thermal heat treatment, at a temperature of about 150° C. to about 280° C. A hot iron may be passed over the hair one or more times.

In various embodiments, the hair treatment composition includes one or more silicones. Silicones can protect hair from damage caused by heat-styling tools such as blow dryers and hot irons. The slick, glossy texture of silicones also helps to impart softness and smoothing to the hair. In various embodiments, at least one of the one or more silicones is an amino-functionalized silicone. Nonlimiting examples of amino-functionalized silicones include amodimethicone, bis-hydroxy/methoxy amodimethicone, bis-cetearyl amodimethicone, bis (C13-15 alkoxy) PG amodimethicone, aminopropyl phenyl trimethicone, aminopropyl dimethicone, bis-amino PEG/PPG-41/3 aminoethyl PG-propyl dimethicone, or a combination thereof.

Film forming polymers may also be incorporated into the hair treatment composition. Film forming polymers can provide additional heat protection to the hair and simultaneously function to “hold” the shape and style of the hair. In various embodiments, the hair treatment composition includes one or more film-forming polymers selected from anionic film-forming polymers, nonionic film-forming polymers, amphoteric film-forming polymers, or a combination thereof.

In various embodiments, the hair treatment composition includes one or more polysaccharides, for example, one or more polysaccharides selected from starches, gums and cellulose-based polymers, and a mixture thereof, preferably one or more polysaccharides are selected from xanthan gum, gellan gum, sclerotium gum, guar gum and its derivatives, cellulose and its derivatives, and a mixture thereof.

In various embodiments, the hair treatment composition includes one or more nonionic associative polymeric thickeners. A nonlimiting but useful class of nonionic associative polymeric thickeners includes polyurethane thickeners, for example, polyurethane/polyether thickeners. Nonlimiting examples of polyurethane/polyether thickeners include PEG-240/HDI copolymer bis-decyltetradeceth-20 ether, PEG-150/stearyl alcohol/SMDI copolymer, PEG-150/decyl alcohol/SMDI copolymer, steareth-100/PEG-136/HDI copolymer, or a mixture thereof.

Hair treated according to the methods disclosed herein exhibits improved fiber alignment, frizz control, and smoothness. These desirable cosmetic attributes are long lasting and are maintained even after multiple washing cycles.

### BRIEF DESCRIPTION OF THE DRAWINGS

Implementation of the present technology is described, by way of example only, with reference to the attached figure, wherein:

The FIGURE shows hair swatches treated according to methods of the instant disclosure and compares them to hair swatches not treated according to the methods of the instant disclosure.

The various aspects of the disclosure are not limited to the results, arrangements, and representations shown in the drawings.

### DETAILED DESCRIPTION OF THE DISCLOSURE

The instant disclosure is drawn to methods for treating hair, especially hair in need of reduced frizz and improved smoothness. This is achieved using a hair treatment composition that includes propylene carbonate. The inventors believe that propylene carbonate undergoes a reaction when subjected to a thermal heat treatment. Heat activates a reaction with the propylene carbon and amine groups of the hair fibers, likely forming an N, N′-disubstituted urea linkage that contributes to the long-lasting fixation of the newly formed shape. The methods are gentle, long-lasting, and reduce hair frizz of while simultaneously improving smoothness. The methods improve fiber alignment, reduce frizz, and impart smoothness, to a surprising degree. The longevity of the improved fiber alignment, reduced frizz, and smoothness is also significant and surprising. Hair treated in accordance with the methods outline herein retains these desirable cosmetic properties, even after multiple cleansing cycles. Thus, the benefits provided by the compositions are surprisingly long-lasting and wash resistant.

Methods in accordance with the instant disclosure typically comprise:
(i) applying a hair treatment composition to hair in need of reduced frizz or increased smoothness; the composition comprising:
   (a) about 1 to about 15 wt. % of propylene carbonate; and
   (b) water,
      wherein the composition is free from alkylene carbonates, cyclic carbonates other than the propylene carbonate, and N-alkyl-2-mercaptoacetamide, and
      all weight percentages are based on a total weight of the composition;
(ii) after rinsing the composition from the hair, subjecting the hair to a heat treatment, wherein the heat treatment heats the hair to a temperature of at least 150° C.

The pH of the composition can vary. Nonetheless, in various embodiments, a pH less than 7 (an acidic pH) is desirable.

### (a) Propylene Carbonate

Propylene carbonate is an organic compound with the formula C₄H₆O₃. It is a cyclic carbonate ester derived from propylene glycol. The hair treatment composition of the instant disclosure typically includes about 1 to about 15 wt. % of propylene carbonate. Nonetheless, in various embodiments, the hair treatment composition includes about 2 to about 15 wt. %, about 3 to about 15 wt. %, about 5 to about 15 wt. %, about 8 to about 15 wt. %, about 1 to about 12 wt. %, about 2 to about 12 wt. %, about 3 to about 12 wt. %, about 5 to about 10 wt. % of propylene carbonate.

The hair treatment composition is typically free or essentially free of carbonates other than the propylene carbonate. For example, the hair treatment composition is typically free from alkylene carbonates and cyclic carbonates other than the propylene carbonate. Furthermore, the hair treatment composition is typically free or essentially free from N-alkyl-2-mercaptoacetamide.

### (b) Water

The hair treatment composition is an aqueous composition, typically containing a substantial amount of water. For example, in various embodiments, the hair treatment composition includes about 50 to about 95 wt. % of water, based on a total weight of the composition. In further embodiments, the compositions include about 60 to about 95 wt. %, about 70 to about 90 wt. %, about 50 to about 85 wt. %, about 60 to about 85 wt. %, about 70 to about 85 wt. %, about 50 to about 80 wt. %, about 60 to about 80 wt. %, about 70 to about 80 wt. %, about 65 to about 85 wt. %, based on the total weight of the compositions.

### (c) Silicones

Nonlimiting examples of silicones include dimethicone, dimethiconol, dimethiconol, cyclomethicone, polysilicone-11, phenyl trimethicone, trimethylsilyl-amodimethicone, and stearoxytrimethylsilane. In various emdobiments, the one or more silicones are non-volatile silicon oils. Useful silicone oils include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone oils that may be mentioned include volatile linear or cyclic silicones, such as those with a viscosity 8 centistokes (8×106 m2/s) and/or containing from 2 to 7 silicon atoms. These silicones optionally comprise alkyl or alkoxy groups containing from 1 to 10 carbon atoms. Non-limiting examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, or mixtures thereof.

In a preferred embodiment, the cosmetic compositions include one or more silicone oils chosen from dimethicone, dimethiconol, cyclomethicone, polysilicone-11, phenyl trimethicone, and amodimethicone, preferably dimethicone.

In an embodiment, cosmetic compositions include one or more amino functionalized silicones. The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, an amino-functionalized silicones is selected from compounds having the following formula:

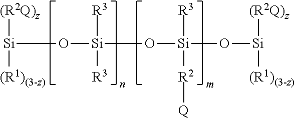

wherein each R¹ is independently selected from a C₁₋₃₀ alkyl group, a C₁₋₃₀ alkoxy group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);
each R² is independently a divalent alkylene radical having one to ten carbon atoms (preferably, R² is a divalent alkylene radical having three to six carbon atoms);
each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);
Q is a monovalent radical selected from —NR⁴₂ and —NR⁴(CH₂)ₓNR⁴₂; each R⁴ is independently selected from a hydrogen and a C₁₋₄ alkyl group;
x is 2 to 6;
z is 0 or 1;
n is 25 to 3,000 (preferably, 25 to 2,000; more preferably, 25 to 1,000; most preferably 25 to 500); and
m is 0 to 3,000 (preferably, 0 to 2,000; more preferably, 0 to 1,000; most preferably, 0 to 100);
with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene, pentamethylene, —CH₂CH(CH₃)CH₂ and CH₂CH₂CH(CH₃)CH₂.

Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicine has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones. Suitable alkoxylated and/or hydroxylated amino silicones may be selected from compounds of the following formula:

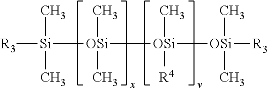

wherein R³ is hydroxyl or OR₅, R₅ is a C₁ to C₄ alkyl group, R₄ is a group with structure according to the following formula:

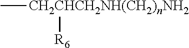

R₆ is a C₁ to C₄ alkyl, n is a 1 to 4, x is the same as “n” described above, and y is the same as “m” described above.

The silicone may be a polysiloxane corresponding to the following formula:

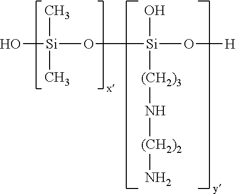

in which x′ and y′ are integers such that the weight-average molecular weight (Mw) is comprised between about 5000 and 500 000;
b) amino silicones corresponding to following formula:

R′ₐG₃₋ₐ—Si(OSiG₂)ₙ-(OSiG_{b}R′_{2-b})ₘ—O—SiG₃₋ₐ—R′ₐ

in which:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula —CqH₂qL in which q is a number ranging from 2 to 8 and L is an optionally quaternized amino group chosen from the following groups:

—NR″-Q-N (R″)₂

—N(R″)₂

—N+(R″)3A-

—NH(R″)2A-

—NH2 (R″A-

—N(R″)-Q-N+R″H₂ A-

—NR″-Q-N+(R″)₂H A-

—NR″-Q-N+(R″)₃ A-,

in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

Another group of amino silicones corresponding to this definition is represented by silicones having the following formula:

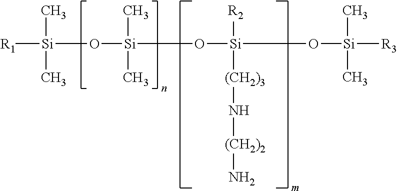

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R¹, R², R³, which may be identical or different, represent a hydroxy or C₁-C₄ alkoxy radical, where at least one of the radicals R¹ to R³ denotes an alkoxy radical.

The alkoxy radical is preferably a methoxy radical. The hydroxy/alkoxy mole ratio ranges preferably from 0.2:1 to 0.4:1 and preferably from 0.25:1 to 0.35:1 and more particularly equals 0.3:1. The weight-average molecular weight (Mw) of the silicone ranges preferably from 2,000 to 1,000,000, more particularly from 3,500 to 200,000.

Another group of amino silicones corresponding to this definition is represented by the following formula:

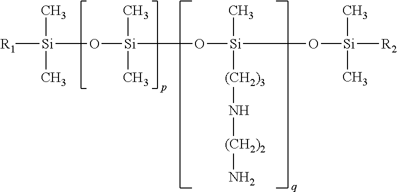

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R¹, R², which may be the same or different, represent a hydroxy or C₁-C₄ alkoxy radical, where at least one of the radicals R¹ or R² denotes an alkoxy radical.

10 The alkoxy radical is preferably a methoxy radical. The hydroxy/alkoxy mole ratio ranges generally from 1:0.8 to 1:1.1 and preferably from 1:0.9 to 1:1 and more particularly equals 1:0.95.

Another group of amino silicones is represented by the following formula:

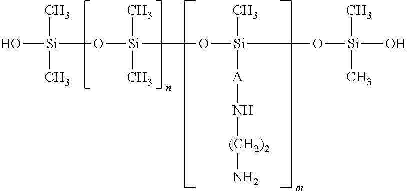

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

Another group of amino silicones is represented by the following formula:

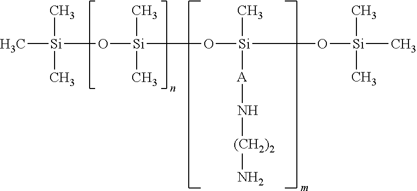

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

Another group of amino silicones is represented by the following formula:

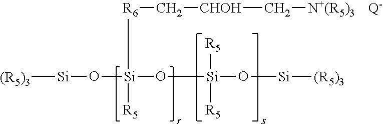

in which:
R⁵ represents a monovalent hydrocarbon-based radical containing from 1 to 18 carbon atoms, and in particular a C₁-C₁₈ alkyl or C₂-C₁₈ alkenyl radical, for example methyl;
R⁶ represents a divalent hydrocarbon-based radical, in particular a C₁-C₁₈ alkylene radical or a divalent C₁-C₁₈, for example C₁-C₈, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in patent U.S. Pat. No. 4,185,087.

A group of quaternary ammonium silicones is represented by the following formula:

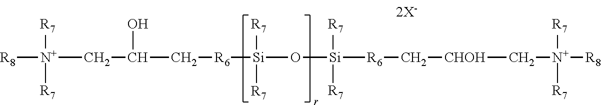

in which:
R⁷, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R⁶ represents a divalent hydrocarbon-based radical, in particular a C₁-C₁₈ alkylene radical or a divalent C₁-C₁₈, for example C₁-C₈, alkylenoxy radical linked to the Si via an SiC bond;
R⁸, which may be identical or different, represent a hydrogen atom, a monovalent hydrocarbon-based radical containing from 1 to 18 carbon atoms, and in particular a C₁-C18 alkyl radical, a C₂-C₁₈ alkenyl radical or a —R⁶-NHCOR⁷ radical;
X- is an anion such as a halide ion, in particular chloride, or an organic acid salt (for example acetate);
r represents a mean statistical value from 2 to 200 and in particular from 5 to 100. These silicones are described, for example, in patent application EP-A 0530974.

A group of quaternary ammonium silicones is represented by the following formula:

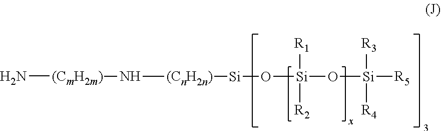

in which:
R¹, R², R³ and R⁴, which may be identical or different, denote a C₁-C₄ alkyl radical or a phenyl group;
R⁵ denotes a C₁-C₄ alkyl radical or a hydroxyl group;
n is an integer ranging from 1 to 5;
m is an integer ranging from 1 to 5;
and in which x is chosen such that the amine number is between 0.01 and 1 meq/g;
multiblockpolyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe₂O)xSiMe₂—R—N(R″)—R′—O(C₂H₄O)ₐ(C₃H₆O)b—R′—N(H)—R—]

or alternatively

[—(SiMe₂O)xSiMe₂—R—N(R″)—R′—O(C₂H₄O)ₐ(C₃H₆O)b—]

in which:
a is an integer greater than or equal to 1, preferably ranging from 5 to 200, more particularly ranging from 10 to 10;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH (OH) CH₂— radical; preferentially R denotes a —CH₂CH₂CH₂OCH(OH)CH₂— radical;
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH (OH) CH₂-radical; preferentially R′ denotes —CH(CH₃)—CH₂—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2. The weight-average molecular weight (Mw) of the silicone oil is preferably comprised between 5000 and 1,000,000, more particularly between 10,000 and 200,000.

Non-limiting examples of amino-functionalized silicones include bis-hydroxy/methoxy amodimethicones, bis-cetearyl amodimethicone, amodimethicone, bis (C₁₃-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl) amino]-2-methylpropyl Me, di-Me, [(hydroxydimethylsilyl)oxy]-and [(methoxydimethylsilyl) oxy]-terminated.” Bis-hydroxy/methoxy amodimethicone is commercially available under the tradename DOWSIL AP-8087 FLUID from The Dow Chemical Company. A particularly preferred amino-functionalized silicone is amodimethicone” A non-limiting example of amodimethicone products containing amino silicones having structure (D) re sold by Wacker under the name BELSIL ADM 652, BELSIL ADM 4000 E, or BELSIL ADM LOG 1. A product containing amino silicones having structure (E) is sold by Wacker under the name FLUID WR 1300. Additionally or alternative, the weight-average molecular weight (Mw) of the silicone ranges preferably from 2,000 to 200,000, even more particularly 5,000 to 100,000 and more particularly from 10,000 to 50,000.

In a preferred embodiment, the one or more amino-functionalized silicones are selected from amodimethicone, bis-hydroxy/methoxy amodimethicone, bis-cetearyl amodimethicone, bis (C₁₃-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In a further preferred embodiments, the amino-functionalized silicone is amodimethicone.

The total amount of the one or more silicones in the compositions will vary. Nonetheless, in various embodiments, the compositions include about 0.1 to about 10 wt. % of the one or more silicones, based on the total weight of the composition. In further embodiments, the compositions include about 0.1 to about 8 wt. %, about 0.1 to about 6 wt. %, about 0.1 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of the one or more silicones, based on the total weight of the composition.

In a preferred embodiment, the hair treatment compositions include one or more amino-functionalized silicones. In further embodiments, the hair treatment compositions include one or more amino-functionalized silicones and are free or essentially free of other silicones, i.e., the composition is free or essentially free from silicones other than the one or more amino-functionalized silicones. In additional embodiments, the hair treatment composition include amodimethicone and is free or essentially from silicones other than the amodimethicone.

The total amount of the one or more amino-functionalized silicones in the compositions will vary. Nonetheless, in various embodiments, the compositions include about 0.1 to about 10 wt. % of the one or more amino-functionalized silicones, based on the total weight of the composition. In further embodiments, the compositions include about 0.1 to about 8 wt. %, about 0.1 to about 6 wt. %, about 0.1 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. %, based on the total weight of the composition.

### (d) Film-Forming Polymers

The hair treatment compositions of the instant disclosure optionally include one or more film-forming polymers, for example, one or more film-forming polymers selected from cationic film-forming polymers, anionic film-forming polymers, nonionic film-forming polymers, amphoteric film-forming polymers, or a combination thereof.

Various types of film-forming polymers overlap with the scope of the one or more polysaccharides (component (e)) and the one or more nonionic associative polymeric thickeners (component (f)). This is because various film-forming polymers, polysaccharides, and nonionic associative polymeric thickeners can contribute to the formation of films on the hair and provide a thickening effect to the hair treatment compositions.

In such cases where overlap may exist and the hair treatment composition includes two or more components that overlap, a single compound does not represent more than one component. For example, if a hair treatment composition includes a film-forming polymer, a polysaccharide, and a nonionic associative thickener, a single compound cannot serve as all three components. Similarly, a single compound cannot simultaneously serve as two components.

In various embodiments, the hair treatment composition includes one or more hydrophilic film-forming polymers. The term “hydrophilic film-forming polymer” is interchangeable with the term “aqueous film-forming polymer” and refers to polymers or copolymers that can be water-soluble or water-dispersible; anionic, cationic, or nonionic; and crosslinked or not cross-linked. They generally include polar groups such as hydroxyl, amide, amine or ether groups, and other groups with a high affinity for water. In addition, they should provide good adhesive, binding, film-forming and water-swelling properties. The hydrophilic polymers are film-forming polymers capable of forming, by themselves or in the presence of an auxiliary film-forming agent, a continuous film capable of adhering to a support, especially to the skin.

Nonlimiting examples of hydrophilic film forming polymers include polyurethanes, vinyl polymers, natural polymers, latex polymers, vinylpyrrolidone (VP)-based polymers, amphoteric polymers, and mixtures thereof.

### (i) Polyurethanes

The polyurethanes may be aliphatic, cycloaliphatic, or aromatic polyurethanes, polyurea-urethanes or polyurea copolymers, comprising, alone or as a mixture: at least one block of aliphatic and/or cycloaliphatic and/or aromatic polyester origin, and/or at least one branched or unbranched silicone block, for example polydimethylsiloxane or polymethylphenylsiloxane, and/or at least one block comprising fluoro groups.

The film-forming polyurethanes that can be used in the invention may also be obtained from branched or non-branched polyesters or from alkyls comprising labile hydrogens, which are modified by reaction with a diisocyanate and a difunctional organic compound (for example dihydroxy, diamino or hydroxyamino), also comprising either a carboxylic acid or carboxylate group, or a sulfonic acid or sulfonate group, or alternatively a neutralizable tertiary amine group or a quaternary ammonium group.

With a view to forming the polyurethane, monomers bearing an anionic group that can be used during the polycondensation that maybe mentioned include dimethylolpropionic acid, trimellitic acid or a derivative such as trimellitic anhydride, the sodium salt of 3-sulfopentanediol acid, and the sodium salt of 5-sulfo-1,3-benzenedicarboxylic acid. Preferably, the monomer bearing an anionic group is dimethylolpropionic acid.

As film-forming polyurethane that may be used according to the invention, mention may thus be made of the aqueous polyurethane dispersions sold under the names AVALURE UR-405®, AVALURE UR-410®, AVALURE UR-425® and AVALURE UR-450® by the company Goodrich. A particularly preferred polyurethane is polyurethane-99.

Advantageously, the film-forming polyurethanes that are selected from copolymers obtained by copolymerization of hexanediol, neopentyl glycol, adipic acid, hexamethylene diisocyanate, N-(2-aminoethyl)-3-aminoethanesulfonic acid and ethylenediamine. Preferably, the polyurethanes may also be selected from copolymers obtained by copolymerization of adipic acid, dicyclohexylmethane diisocyanate, ethylenediamine, hexanediol, neopentyl glycol and sodium N-(2-aminoethyl)-3-aminoethanesulfonate.

In particular, the polyurethanes are chosen from those sold under the name BAYCUSAN ECO E 1001, BAYCUSAN C₁₀₀₁ or C₁₀₀₄, known as polyurethane-99, polyurethane-35, and more particularly the product sold under the name BAYCUSAN C₁₀₀₁, known as polyurethane-99.

### (ii) Vinyl Polymers

Vinyl polymers may be chosen from polyvinyl alcohols, copolymers derived from C₄-C₈ monounsaturated carboxylic acids or anhydrides, and methyl vinyl ether/butyl monomaleate copolymers. For the purpose of the present invention, the term “polyvinyl alcohol” means a polymer comprising —CH₂CH(OH)— units. The polyvinyl alcohols are generally produced by hydrolysis of polyvinyl acetate. Usually, the reaction takes place in the presence of methanol (alcoholysis). The reaction is normally catalyzed by acidic or basic catalysis. The degree of hydrolysis of the commercial products is variable, often around 87%, but products with a 100 degree of hydrolysis also exist. Copolymers with monomers other than vinyl acetate also exist, such as ethylene/vinyl alcohol copolymers.

The polyvinyl alcohol polymers are preferably chosen from homopolymers or copolymers with vinyl acetate, the latter corresponding in particular to a partial hydrolysis of polyvinyl acetate.

Use may, for example, be made of the products of the CELVOL range provided by the company Celanese under the names CELVOL 540, CELVOL 350, CELVOL 325, CELVOL 165, CELVOL 125, CELVOL 540 S, CELVOL 840 and CELVOL 443.

The copolymer(s) derived from C₄-C₈ monounsaturated carboxylic acids or anhydrides may be chosen from copolymers comprising (i) one or more maleic, fumaric or itaconic acids or anhydrides and (ii) one or more monomers chosen from vinyl esters, vinyl ethers, vinyl halides, phenylvinyl derivatives, and acrylic acid and its esters, the anhydride functions of these copolymers being optionally monoesterified or monoamidated, for example, INCI name: Butyl Ester of PVM/MA Copolymer. Preferably, the copolymer(s) derived from C₄-C₈ monounsaturated carboxylic acids or anhydrides are chosen from the monoesterified methyl vinyl ether/maleic anhydride copolymers, for example, ethyl ester of PVM/MA copolymer, sold under the name GANTREZ ES 225 by the company ISP.

(iii) Natural Polymers

The hydrophilic polymers may also be chosen from natural polymers, in particular polysaccharides which have monosaccharides or disaccharides as base units. The natural polymers are preferably chosen from pullulan, guar gums and modified guar gums, celluloses, and gellan gum, and derivatives thereof.

In a preferred embodiment, a natural hydrophilic film-forming polymer is pullulan. Pullulan is a polysaccharide polymer consisting of maltotriose units, also known as α-1,4-; α-1,6-glucan'. Three glucose units in maltotriose are connected by an α-1,4 glycosidic bond, whereas consecutive maltotriose units are connected to each other by an α-1,6 glycosidic bond. In various embodiments, the skin perfecting compositions include pullulan and optionally one or more additional hydrophilic film-forming polymers, for example, one or more polyurethanes. In such situation, the amount of the pullulan will vary. Nonetheless, in various embodiments, the skin perfecting compositions includes about 1 to about 15 wt. % of the pullulan, based on a total weight of the skin perfecting composition. In further embodiments, the skin perfecting composition includes about 1 to about 15 wt. %, about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 8 wt. %, of pullulan based on a total weight of the skin perfecting composition.

Guar gums are galactomannans consisting of mannose and galactose. For the purpose of the present disclosure, the term “modified guar gum” means guar gums alkylated with at least one C₁-C₈ alkyl group, guar gums hydroxyalkylated with at least one C₁₋₈ hydroxyalkyl group and guar gums acylated with at least one C₁₋₈ acyl group. Hydroxypropylated guar gums (e.g., hydroxypropyl guar) such as the product sold under the name JAGUAR HP 105 by the company Rhodia is a useful example.

The cellulose is a 1-4-polyacetal of cellobiose, cellobiose being a disaccharide consisting of two glucose molecules. The cellulose derivatives may be cationic, amphoteric or nonionic. Among these derivatives, cellulose ethers, cellulose esters and cellulose ester ethers are distinguished. Among the nonionic cellulose ethers, mention may be made of alkylcelluloses such as methylcelluloses and ethylcelluloses; hydroxyalkylcelluloses such as hydroxymethylcelluloses, hydroxyethylcelluloses and hydroxypropylcelluloses; and mixed hydroxyalkyl-alkylcelluloses such as hydroxypropylmethylcelluloses, hydroxyethylmethylcelluloses, hydroxyethylethylcelluloses and hydroxybutylmethylcelluloses.

Among the cationic cellulose ethers, mention may be made of crosslinked or non-crosslinked quaternized hydroxyethylcelluloses. The quaternizing agent may especially be glycidyltrimethylammonium chloride or a fatty amine such as laurylamine or stearylamine. Another cationic cellulose ether that may be mentioned is hydroxyethylcellulosehydroxypropyltrimethylammonium. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates. The cellulose-based compounds of the invention may be chosen from unsubstituted celluloses and substituted celluloses.

The celluloses and derivatives are represented, for example, by the products sold under the names AVICEL® (microcrystalline cellulose, MCC) by the company FMC Biopolymers, under the name METHOCEL™ (cellulose ethers) and ETHOCEL™ (ethylcellulose) by the company Dow, BENECEL® (methylcellulose), BLANOSE™ (carboxymethylcellulose), CULMINAI® (methylcellulose, hydroxypropylmethylcellulose), KLUCEL® (hydroxypropylcellulose), POLYSURF® (cetylhydroxyethylcellulose) and NATROSOL® CS (hydroxyethylcellulose) by the company Hercules Aqualon.

Gellan gum is a polysaccharide produced by aerobic fermentation of Sphingomonas elodea, more commonly known as Pseudomonas elodea. This linear polysaccharide is formed from the sequence of the following monosaccharides: D-glucose, D-glucuronic acid and L-rhamnose. In native form, gellan gum is highly acylated. The gellan gum preferably used in the film according to the present invention is a gellan gum that is at least partially deacylated. This at least partially deacylated gellan gum is obtained by high-temperature alkaline treatment. A solution of KOH or of NaOH will, for example, be used. The purified gellan gum sold under the trade name KELCOGEL® by the company Kelco is suitable for preparing the compositions according to the invention.

Gellan gum derivatives are all the products obtained by performing standard chemical reactions, especially such as esterifications, addition of a salt of an organic or mineral acid. Welan gum is used, for example, as a gellan gum derivative. Welan gum is a gellan gum modified by fermentation by means of Alcaligenes strain ATCC 31 555. Welan gum has a recurring pentasaccharide structure formed from a main chain consisting of D-glucose, D-glucuronic acid and L-rhamnose units, onto which a pendent L-rhamnose or L-mannose unit is grafted. The welan gum (diutan gum) sold under the trade name KELCO CRETE® by the company Kelco is suitable for preparing the compositions according to the invention.

As other saccharide polymers that can be used according to the invention, mention may be made of starches and derivatives thereof.

Natural hydrophilic film-forming polymers include celluloses and derivatives thereof, in particular those sold under the name AVICEL® (microcrystalline cellulose, MCC) by the company FMC Biopolymers.

Carrageenans are anionic polysaccharides constituting the cell walls of various red algae (Rhodophyceae) belonging to the Gigartinacae, Hypneaceae, Furcellariaceae and Polyideaceae families. They are generally obtained by hot aqueous extraction from natural strains of said algae. These linear polymers, formed by disaccharide units, are composed of two D-galactopyranose units linked alternately by α(1,3) and β(1,4) bonds. They are highly sulfated polysaccharides (20%-50%) and the α-D-galactopyranosyl residues may be in 3,6-anhydro form. Depending on the number and position of sulfate-ester groups on the repeating disaccharide of the molecule, several types of carrageenans are distinguished, namely: kappa-carrageenans, which bear one sulfate-ester group, iota-carrageenans, which bear two sulfate-ester groups, and lambda-carrageenans, which bear three sulfate-ester groups. Carrageenans are composed essentially of potassium, sodium, magnesium, triethanolamine and/or calcium salts of polysaccharide sulfate esters.

Carrageenans are sold especially by the company SEPPIC under the name SOLAGUM®, by the company Gelymar under the names CARRAGEL®, CARRALACT® and Carrasol®, by the company Cargill under the names Satiagel™ and SATIAGUM™, and by the company CP-Kelco under the names GENULACTAR, GENUGEL® and GENUVISCO®.

Mention is made of hyaluronic acid and salts therefore, for example sodium hyaluronate and potassium hyaluronate. Sodium hyaluronate is the sodium salt of hyaluronic acid. It is a glycosaminoglycan and long-chain polymer of disaccharide units of Na-glucuronate-N-acetylglucosamine.

A useful hydrophilic film forming polymer is xanthan gum and modified xanthan gums, such as dehydroxanthan gum, hydroxypropyl xanthan gum, and mixtures thereof. In some cases, dehydroxanthan gum is useful.

### (iv) Latex or Pseudolatex Polymers

As presented above for the hydrophilic film-forming polyurethanes, the hydrophilic film-forming polymer may thus also be present in a composition of the invention in the form of particles dispersed in an aqueous phase, which is generally known as a latex or pseudolatex. Techniques for preparing these dispersions are well known to those skilled in the art.

Aqueous dispersions of film-forming polymers that may be used include the acrylic dispersions sold under the names NEOCRYL XK-90, NEOCRYL A-1070®, NEOCRYL A-1090R, NEOCRYL BT-62®, NEOCRYL A-1079® and NEOCRYL A-523® by the company Avecia-Neoresins, Dow Latex 432® by the company Dow Chemical, DAITOSOL 5000 ADR or DAITOSOL 5000 SJ® by the company Daito Kasey Kogyo; SYNTRAN 5760® or SYNTRAN PC 5100@ by the company Interpolymer, Allianz OPT by the company Rohm & Haas, aqueous dispersions of acrylic or styrene/acrylic polymers sold under the brand name JONCRYL® by the company Johnson Polymer, or the aqueous dispersions of polyurethane sold under the names NEOREZ R-981® and NEOREZ R-974® by the company Avecia-Neoresins, AVALURE UR-405®, AVALURE UR-410®, AVALURE UR-425®, AVALURE UR-450®, SANCURE 875®, SANCURE 861®, SANCURE 878® and SANCURE 2060@ by the company Goodrich, IMPRANIL 85@ by the company Bayer and AQUAMERE H-1511® by the company Hydromer; the sulfopolyesters sold under the brand name EASTMAN AQQ by the company Eastman Chemical Products, and vinyl dispersions, for instance MEXOMER PAM® from the company Chimex, and mixtures thereof.

### (v) Vinylpyrrolidone (VP)-Based Polymers

Nonlimiting examples of vinylpyrrolidone (VP)-based film-forming polymers include polyvinylpyrrolidone (PVP), VP-styrene copolymer, VP-vinyl acetate copolymer, and diethyl sulfate VP-dimethylaminoethyl-methacrylic acid copolymer, and blends thereof.

### (vi) Amphoteric Polymers

Nonlimiting examples of hydrophilic film-forming polymers include amphoteric polymers. Nonlimiting examples include polymethacryloyloxyethyltrimethyl ammonium chloride, alkyl vinyl ether maleic anhydride (AVE/MA) copolymer, poly-2-aminopropyl acrylate, poly (diethylaminoethyl methacrylate), copolymers such as dimethylaminoethyl methacrylate copolymer and zwitterionic polymers such as polybetaines such as poly-2-ethynyl-N-(4-sulfobutyl) pyridinium betaine (PESPB), polysulfobetaines such as poly-N, N-dimethyl-N-3-sulfopropyl-3′methacrylamidopropanaminium and copolymers such as diallyldimethylammonium chloride-maleamic acid copolymers, or a combination thereof.

The amount of the one or more film-forming polymers in the hair treatment compositions, if present, will vary. Nonetheless, in various embodiments, the hair treatment composition includes about 0.1 to about 10 wt. % of the one or more film-forming polymers, based on a total weight of the hair treatment composition. In further embodiments, the hair treatment composition includes about 0.1 to about 8 wt. %, about 0.1 to about 6 wt. %, about 0.1 to about 5 wt. %, about 0.1 to about 3 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. %, based on a total amount of the hair treatment composition.

### (e) Polysaccharides

The term “polysaccharides” refers to compounds containing a backbone of repeating sugar (i.e., carbohydrate) units. The polysaccharides may be cationic, nonionic, or anionic. In various embodiments, the polysaccharides are preferably nonionic polysaccharides.

Nonlimiting examples of polysaccharides include starches, gums, celluloses, and a mixtures thereof. Nonlimiting examples of starches include modified starches, starch-based polymers, methylhydroxypropyl starch, potato starch, wheat starch, rice starch, starch crosslinked with octenyl succinic anhydride (sold under the name Dry-Flo by National Starch), starch oxide, dialdehyde starch, dextrin, British gum, acetyl starch, starch phosphate, carboxymethyl starch, hydroxyethyl starch, and hydroxypropyl starch.

Nonlimiting examples of cellulose-based polymers include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename NATROSOL CS Plus from Aqualon Corporation.

In various embodiments, the polysaccharides are preferably selected from scleroglucans comprising a linear chain of (1-3) linked glucose units with a (1-6) linked glucose every three units, a commercially available example of which is CLEAROGEL. CS1 1 from Michel Mercier Products Inc.

Nonlimiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof.

In various embodiments, the one or more polysaccharides are selected from hydroxypropyl starch phosphate, potato starch (modified or unmodified), wheat starch, rice starch, hydroxyethyl cellulose, guar gum, hydroxypropyl guar, xanthan gum, sclerotium gum, and a mixture thereof. In yet further embodiments, the polysaccharide is hydroxypropyl starch phosphate. Hydroxypropyl starch phosphate is sold under the tradename of STRUCTURE ZEA by the company Akzo Nobel. In a preferred embodiment, at least one of the one or more polysaccharides is sclerotium gum.

The total amount of the one or more polysaccharides in the compositions will vary. Nonetheless, in various embodiments, the compositions include about 0.1 to about 8 wt. % of the one or more polysaccharides, based on the total weight of the composition. In further embodiments, the compositions include about 0.1 to about 6 wt. %, about 0.1 to about 5 wt. %, about 0.1 to about 4 wt. %, about 0.1 to about 3 wt. %, about 0.1 to about 2 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.3 to about 8 wt. %, about 0.3 to about 6 wt. %, about 0.3 to about 5 wt. %, about 0.3 to about 5 wt. %, about 0.3 to about 4 wt. %, about 0.3 to about 3 wt. %, or about 0.3 to about 2 wt. %, based on the total weight of the composition.

### (f) Nonionic Associative Polymeric Thickeners

An associative thickener is capable of reversibly associating with itself or with other molecules or particles. This physical association provides thickening and can give rise to thixotropic or shear-thinning macromolecular systems, i.e. systems whose viscosity depends on the shear forces to which they are subjected.

In various embodiments, the nonionic associative polymer thickener is a nonionic associative polyurethane thickener, preferably a nonionic associative polyurethane/polyether. The nonionic polyurethane/polyethers may have both at least one hydrophilic moiety and at least one hydrophobic moiety. More particularly, said polymers may contain in their chain both hydrophilic sequences most often of a polyoxyethylenated nature and hydrophobic sequences which may be aliphatic linkages alone and/or cycloaliphatic and/or aromatic linkages. In various embodiments, these polyether-polyurethanes comprise at least two lipophilic hydrocarbon chains, having from 6 to 30 carbon atoms, preferably from 6 to 20, separated by a hydrophilic sequence, it being possible for the hydrocarbon chains to be pendent chains or chains at the end of a hydrophilic sequence. In particular, it is possible for one or more pendent chains to be envisaged. In addition, the polymer may comprise a hydrocarbon chain at one end or at both ends of a hydrophilic sequence.

The polyether-polyurethanes may be polyblocks, in particular in triblock form. The hydrophobic sequences may be at each end of the chain (for example: triblock copolymer with hydrophilic central sequence) or distributed both at the ends and in the chain (polyblock copolymers for example). These same polymers may also be in the form of graft units or may be star shaped.

The nonionic polyether/polyurethanes containing a fatty chain may be triblock copolymers whose hydrophilic sequence is a polyoxyethylenated chain comprising from 50 to 1000 oxyethylenated groups. The nonionic polyether-polyurethanes comprise a urethane bond between the hydrophilic sequences, hence the origin of the name. By extension, those whose hydrophilic sequences are linked by other chemical bonds to the hydrophobic sequences are also included among the nonionic polyether-polyurethanes containing a hydrophobic chain.

Nonlimiting examples of nonionic polyether/polyurethanes containing a hydrophobic chain include Rheolate® 205 containing a urea functional group sold by the company RHEOX or else the Rheolates® 208, 204 or 212, as well as Acrysol RM 184®. Additional products include ELFACOS T210® containing a C₁₂-C₁₄ alkyl chain and the product ELFACOS T212® containing a C₁₈ alkyl chain from AKZO. The product DW 1206B® from ROHM & HAAS containing a C₂₀ alkyl chain and with a urethane bond, sold at 20% dry matter content in water, may also be used.

It is also possible to use solutions or dispersions of these polymers in particular in water or in an aqueous-alcoholic medium. By way of examples of such polymers, there may be mentioned Rheolate® 255, Rheolate® 278 and Rheolate® 244 sold by the company RHEOX. It is also possible to use the product DW 1206F and DW 1206J provided by the company ROHM & HAAS.

As the above-described polyether/polyurethanes, mention may be made of polyurethane/polyethers comprising in their chain at least one polyoxyethylenated hydrophilic block and at least one of hydrophobic blocks containing at least one sequence chosen from aliphatic sequences, cycloaliphatic sequences, and aromatic sequences. In various embodiments, it is preferable that the polyurethane/polyethers comprise at least two hydrocarbon-based lipophilic chains having from 8 to 30 carbon atoms, separated by a hydrophilic block, and wherein the hydrocarbon-based chains are chosen from pendent chains and chains at the end of the hydrophilic block.

In a preferred embodiment, use is made a polyurethane/polyether that may be obtained by polycondensation of at least three compounds comprising (i) at least one polyethylene glycol comprising from 150 to 180 mol of ethylene oxide, (ii) a polyoxyethylenated stearyl alcohol comprising 100 mol of ethylene oxide, and (iii) a diisocyanate. Such polyurethane/polyethers are sold especially by the company Element is under the name Rheolate FX 1100® and Rheoluxe 811®, which is a polycondensate of polyethylene glycol containing 136 mol of ethylene oxide, of stearyl alcohol polyoxyethylenated with 100 mol of ethylene oxide and of hexamethylene diisocyanate (HDI) with a weight-average molecular weight of 40000 (INCI name: PEG-136/Steareth-100/HDI Copolymer).

According to another embodiment, use will be made of a polyurethane/polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate. Such polyurethane/polyethers are sold in particular by the company Rohm & Haas under the names Aculyn 46® and Aculyn 44®.

Aculyn 46® having the INCI name: PEG-150/Stearyl Alcohol/SMDI Copolymer, is a polycondensate of polyethylene glycol comprising 150 or 180 mol of ethylene oxide, of stearyl alcohol and of methylenebis (4-cyclohexyl isocyanate) (SMDI) at 15% by weight in a matrix of maltodextrin (4%) and water (81%) (INCI name: PEG-150/Stearyl Alcohol/SMDI Copolymer). Aculyn 44® (PEG-150/Decyl Alcohol/SMDI Copolymer) is a polycondensate of polyethylene glycol comprising 150 or 180 mol of ethylene oxide, of decyl alcohol and of methylenebis (4-cyclohexyl isocyanate) (SMDI) at 35% by weight in a mixture of propylene glycol (39%) and water (26%) (INCI name: PEG-150/Decyl Alcohol/SMDI Copolymer).

As the associative polyurethanes, it may be preferable to use a compound represented by the following formula (1):

R¹—{(O—R²)ₖ—OCONH—R³[—NHCOO—(R⁴—O)—R⁵]ₕ}ₘ (1)
wherein R¹ represents a hydrocarbon group, R² and R⁴ independently represent alkylene groups having 2 to 4 carbon atoms, which alkylene groups may be identical or different from each other, or a phenylethylene group, R³ represents a hydrocarbon group, which may optionally have a urethane bond, R⁵ represents a branched chain or secondary hydrocarbon group, “m” represents a number of at least 2, “h” represents a number of at least 1, “k” represents a number within the range of 1 to 500, and “n” represents a number within the range of 1 to 200.

The hydrophobically modified polyurethane represented by the general formula (1) shown above is obtained by, for example, reacting at least one polyether polyol that is represented by the formula R¹—[(O—R²)ₖ—OH]ₘ, at least one polyisocyanate that is represented by the formula R³—(NCO)ₕ₊₁, and at least one polymonoalcohol that is represented by the formula HO—(R⁴—O)ₙ—R⁵. In such cases, R¹ to R⁵ in the general formula (1) are determined by the compounds R¹—[(O—R²)ₖ—OH]ₘ, R³—(NCO)ₕ₊₁ and HO—(R⁴—O)ₙ—R⁵. The loading ratios among the three compounds are not limited particularly and should preferably be such that the ratio of the isocyanate group derived from the polyisocyanate to the hydroxyl group derived from the polyether polyol and the polyether monoalcohol is selected within the range of NCO/OH of between 0.8:1 and 1.4:1.

The polyether polyol compound that is represented by the formula R¹—[(O—R²)ₖ—OH]ₘ and that may be used preferably for obtaining the associative thickener represented by the general formula (1) may be obtained from addition polymerization of an m-hydric polyol with an alkylene oxide, such as ethylene oxide, propylene oxide, butylene oxide, or epichlorohydrin, or with styrene oxide, and the like.

The polyols should preferably be di-to octa-hydric polyols. Examples of the di-to octa-hydric polyols include dihydric alcohols, such as ethylene glycol, propylene glycol, butylene glycol, hexamethylene glycol, and neopenthyl glycol; trihydric alcohols, such as glycerol, trioxy isobutane, 1,2,3-butanetriol, 1,2,3-pentanetriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, pentamethylglycerol, pentaglycerol, 1,2,4-butanetriol, 1,2,4-pentanetriol, trimethylolethane, and trimethylolpropane; tetrahydric alcohols, such as pentaerythritol, 1,2,3,4-pentanetetrol, 2,3,4,5-hexanetetrol, 1,2,4,5-pentanetetrol, and 1,3,4,5-hexanetetrol; pentahydric alcohols, such as adonitol, arabitol, and xylitol; hexahydric alcohols, such as dipentaerythritol, sorbitol, mannitol, and iditol; and octahydric alcohols, such as sucrose.

Also, R² is determined by the alkylene oxide, styrene oxide, or the like, which is subjected to the addition. Particularly, for availability and excellent effects, an alkylene oxide having 2 to 4 carbon atoms, or styrene oxide is preferable.

The alkylene oxide, styrene oxide, or the like, to be subjected to the addition may be subjected to single polymerization, or random polymerization or block polymerization of at least two members. The procedure for the addition may be a conventional procedure. Also, the polymerization degree K may be selected within the range of 0 to 1,000, preferably within the range of 1 to 500, and more preferably within the range of 10 to 200. Further, the ratio of the ethylene group occupying R² should preferably be within the range of 50 to 100 mass % with respect to the total quantity of R^{2.} In such cases, the associative thickener appropriate for the purposes of the present invention is obtained.

Furthermore, the molecular weight of the polyether polyol compound that is represented by the formula R¹-[(O-R²)ₖ—OH]ₘ, should preferably be selected within the range of 500 to 100,000, and should more preferably be selected within the range of 1,000 to 50,000.

The polyisocyanate that is represented by the formula R³-(NCO) h+1 and that may be used preferably for obtaining the hydrophobically modified polyether urethane represented by the general formula (1) employed in accordance with the present invention is not limited particularly in so far as the polyisocyanate has at least two isocyanate groups in the molecule. Examples of the polyisocyanates include aliphatic diisocyanates, aromatic diisocyanates, alicyclic diisocyanates, biphenyl diisocyanate, phenylmethane diisocyanate, phenylmethane triisocyanate, and phenylmethane tetraisocyanate.

Also, it is possible to employ dimers and trimers (isocyanurate bonds) of the above-enumerated polyisocyanates. Further, it is possible to employ biuret obtained by a reaction with an amine.

Furthermore, it is possible to employ a polyisocyanate having a urethane bond obtained by a reaction of the aforesaid polyisocyanate compound and a polyol. As the polyol, di-to octa-hydric polyols are preferable, and the above-enumerated polyols are preferable. In cases where a tri-or higher-hydric polyisocyanate is used as the polyisocyanate that is represented by the formula R³-(NCO)n+1, it is preferable to employ the aforesaid polyisocyanate having the urethane bond.

The polyether monoalcohol that is represented by the formula HO-(R⁴-O)ₙ-R5 and that may be used preferably for obtaining the hydrophobically modified polyether urethane represented by the general formula (1) employed in accordance with the present invention is not limited particularly in so far as the polyether monoalcohol is a polyether of a straight chain, branched chain, or secondary monohydric alcohol. The polyether monoalcohol may be obtained by addition polymerization of the straight chain, branched chain, or secondary monohydric alcohol with an alkylene oxide, such as ethylene oxide, propylene oxide, butylene oxide, or epichlorohydrin, or with styrene oxide, and the like.

The compound represented by the general formula (1) may be produced by, for example, heating at a temperature of 80 to 90° C. for 1 to 3 hours and thereby causing a reaction to occur in the same manner as that in the ordinary reaction of a polyether and an isocyanate.

As the compound represented by the general formula (1), polyethyleneglycol-240/decyltetradeceth-20/hexamethylene diisocyanate copolymer is preferable. The polyethyleneglycol-240/decyltetradeceth-20/hexamethylene diisocyanate copolymer is referred to also as PEG-240/HDI copolymer bis-decyltetradeceth-20 ether.

In various embodiments, it is preferable that the nonionic associative polyurethane thickener be selected from steareth-100/PEG-136/HDI copolymer sold by the company Rheox under the name of Rheolate FX 1100, PEG-240/HDI copolymer bis-decyltetradeceth-20 ether sold by the company Asahi Denka under the name of Adekanol GT-700, and mixtures thereof.

The total amount of the one or more nonionic associative polymeric thickeners will vary. Nonetheless, in various embodiments, the compositions include about 0.1 to about 8 wt. %, based on the total weight of the compositions. In further embodiments, the compositions include about 0.1 to about 5 wt. %, about 0.1 to about 4 wt. %, about 0.1 to about 3 wt. %, about 0.1 to about 2 wt. %, about 0.5 to about 8 wt. %, about 0.5 to about 5 wt. %, about 0.5 to about 4 wt. %, about 0.5 to about 3 wt. %, about 0.5 to about 2 wt. %, based on the total weight of the compositions.

### (g) Nonionic Surfactants or Emulsifiers

The terms “nonionic surfactant” and “nonionic emulsifier” are used interchangeably in the instant disclosure and therefore can be referred to as “nonionic emulsifying surfactants.” The nonionic surfactant or emulsifier may have an HLB (hydrophilic-lipophilic balance) ranging from 1 to 7.9 or greater than or equal to 8. “HLB” refers to the “hydrophilic-lipophilic balance” associated with nonionic surfactants or emulsifiers. In particular, “HLB” value relates to the ratio of hydrophilic groups and lipophilic groups in emulsifiers, and also relates to solubility of the emulsifiers. Lower HLB emulsifiers (such as those with HLB values ranging from 1 to 7.9) are more soluble in oils (lipophilic material) and are more appropriate for use in water-in-oil (W/O) emulsions. Higher HLB emulsifiers (such as those with HLB values higher than 8) are more soluble in water (hydrophilic material) and are more appropriate for oil-in-water

(O/W) emulsions.

Nonlimiting examples of nonionic surfactants or emulsifiers include alkyl and polyalkyl esters of poly (ethylene oxide), alkyl and polyalkyl ethers of poly (ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Preferably, the non-ionic surfactant(s) may be chosen from alkyl and polyalkyl esters of poly (ethylene oxide), alkyl and polyalkyl ethers of poly (ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and mixtures thereof.

(1) Alkyl and polyalkyl esters of poly (ethylene oxide) that are preferably used are those containing at least one C₈-C₃₀ alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate,

PEG-10 oleate and PEG-40 hydrogenated castor oil.

(2) Alkyl and polyalkyl ethers of poly (ethylene oxide) that are preferably used are those containing at least one C₈-C₃₀ alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C₁₂-15 pareth-3, C₁₂-13 pareth-4, C₁₂-13 pareth-23, trideceth-3, trideceth-4, trideceth-5, trideceth-6, trideceth-7 and trideceth-10, and mixtures thereof.

(3) Polyoxyethylenated alkyl and polyalkyl esters of sorbitan that are preferably used are those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

(4) Polyoxyethylenated alkyl and polyalkyl ethers of sorbitan that are preferably used are those with a number of ethylene oxide (EO) units ranging from 0 to 100.

The compositions of the instant disclosure may include one or more alkanolamides. Non-limiting examples alkanolamides include fatty acid alkanolamides. The fatty acid alkanolamides may be fatty acid monoalkanolamides or fatty acid dialkanolamides or fatty acid isoalkanolamides, and may have a C₂-8 hydroxyalkyl group (the C₂-8 chain can be substituted with one or more than one-OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

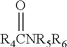

wherein R⁴ is an alkyl chain of 4 to 20 carbon atoms (R⁴ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof);

R⁶ is selected from —CH₂OH, —CH₂CH₂OH, —CH₂CH₂CH₂OH,-

CH₂ (CHOH) 4CH₂OH, -benzyl, and mixtures thereof;

R⁶ is selected from —H, —CH₃, —CH₂OH, —CH₂CH₃, —CH₂CH₂OH, —CH₂CH₂CH₂OH, —CH₂ (CHOH)₄CH₂OH, -benzyl, and mixtures thereof.

In some instances, the one or more of the fatty acid alkanolamides include one or more acyl glucamides, for example, acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The compositions of the instant disclosure may include one or more alkyl polyglucosides. Non-limiting examples of alkyl polyglucosides include those having the following formula:

R¹—O—(R²O)ₙ—Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Useful alkyl poly glucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

The compositions of the instant disclosure may include one or more miscellaneous nonionic surfactants or emulsifiers. Nonlimiting examples include alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols;

polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄) alkylpolyglycosides; N-(C₆-C₂₄) alkylglucamine derivatives, amine oxides such as (C₁₀-C₁₄) alkylamine oxides or N-(C₁₀-C14) acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C24, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di-and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di-and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

The total amount of the one or more nonionic surfactants or emulsifiers in the compositions, I present, will vary. Nonetheless, in various embodiments, the compositions include about 0.1 to about 10 wt. % of the one or more nonionic surfactants or emulsifiers. In further embodiments, the compositions include about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. %, based on the total weight of the compositions.

### (h) Water-Soluble Organic Solvents

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from glycerin, alcohols (for example, C₁₋₁₀, C₁₋₈, or C₁₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the composition iinclude one or more glycols selected from glycerin, propylene glycol, butylene glycol, pentylene glycol, hexylene glycol, caprylyl glycol, dipropylene glycol, and mixtures thereof.

The total amount of the one or more water soluble solvents in the compositions, if present, will vary. Nonetheless, in various embodiments, the compositions include about 0.1 to about 20 wt. % of the one or more water soluble solvents, based on the total weight of the compositions. In further embodiments, the compositions include about 0.1to about 15 wt. %, about 0.1 to about 10 wt. %, about 0.5 to about 20 wt. %, about 0.5 to about 15 wt. %, about 0.5 to about 10 wt. %, about 1 to about 20 wt. %, about 1 to about 15 wt. %, about 1 to about 10 wt. %, about 2 to about 20 wt. %, about 2 to about 15 wt. %, about 2 to about 10 wt. %, about 5 to about 20 wt. %, about 5 to about 15 wt. %, or about 5 to about 10 wt. %, based on the total weight of the compositions.

### (i) Non-Silicone-based Fatty Compounds

The term “fatty substance” means an organic compound without silicone that is insoluble in water at ordinary temperature (25° C.) and at atmospheric pressure (760 mmHg), i.e. which has a solubility of less than 5%, preferably less than 1% and even more preferentially less than 0.1%. They have in their structure a hydrocarbon-based chain containing at least 6 carbon atoms.

More particularly, the one or more non-silicone-based fatty compounds may be selected from C₆-C₁₆ hydrocarbons, hydrocarbons containing more than 16 carbon atoms, non-silicone oils of animal origin, plant oils of triglyceride type, synthetic triglycerides, fluoro oils, fatty alcohols, non-salified fatty acids, fatty acid and/or fatty alcohol esters other than triglycerides and plant waxes, non-silicone waxes and silicones, and mixtures thereof.

Fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ hydrocarbons, they are linear, branched or optionally cyclic, and are preferably alkanes. Examples that may be mentioned include hexane, dodecane and isoparaffins such as isohexadecane and isodecane.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

The linear or branched hydrocarbons of mineral or synthetic origin containing more than 16 carbon atoms are preferably chosen from liquid paraffins, petroleum jelly, liquid petroleum jelly, polydecenes and hydrogenated polyisobutene such as Parleam®.

The fluoro oils may be chosen from perfluoromethylcyclopentane and perfluoro-1,3-dimethylcyclohexane, sold under the names Flutec® PC₁ and Flutec® PC₃ by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols that may be used in the cosmetic composition of step a) are saturated or unsaturated, linear or branched alcohols comprising from 6 to 30 carbon atoms and more particularly from 8 to 30 carbon atoms, among which mention may be made, for example, of cetyl alcohol, stearyl alcohol and the mixture thereof (cetylstearyl alcohol or cetearyl alcohol), octyldodecanol, 2-butyloctanol, 2-hexyldecanol, 2-undecylpentadecanol, oleyl alcohol or linoleyl alcohol.

The non-salified fatty acids that may be used in the cosmetic composition of step a) may be saturated or unsaturated carboxylic acids comprising from 6 to 30 carbon atoms and in particular from 9 to 30 carbon atoms. They are more particularly chosen from myristic acid, palmitic acid, stearic acid, behenic acid, oleic acid, linoleic acid, linolenic acid and isostearic acid.

These acids are not salified. This means that they are introduced in the form of free acids and that the composition does not comprise any alkaline agent leading to their salification.

The esters of fatty acids and/or of fatty alcohols, advantageously different from the triglycerides mentioned above, which may be used in the cosmetic composition used in step a) are esters of saturated or unsaturated, linear or branched C1-C26 aliphatic mono-or polyacids and of saturated or unsaturated, linear or branched C1-C26 aliphatic mono-or polyalcohols, the total carbon number of the esters more particularly being greater than or equal to 10. Among the monoesters, mention may be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso) stearyl octanoate; isocetyl octanoate; octyl octanoate; cetyl octanoate; decyl oleate;

isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C4-C22 dicarboxylic or tricarboxylic acids and of C1-C22 alcohols and esters of mono-, di-or tricarboxylic acids and of C2-C26 di-, tri-, tetra-or pentahydroxy alcohols may also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate;

pentaerythrityl tetraisostearate; pentaerythrityl tetraoctanoate; propylene glycol dicaprylate; propylene glycol dicaprate, tridecyl erucate; triisopropyl citrate; triisostearyl citrate; glyceryl trilactate; glyceryl trioctanoate; trioctyldodecyl citrate; trioleyl citrate, propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The esters according to this variant may also be chosen from monoesters, diesters, triesters, tetraesters and polyesters, and mixtures thereof. These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters. More particularly, use is made of monoesters and diesters and in particular mono- or di-oleate, -stearate, -behenate, -oleopalmitate, -linoleate, -linolenate or -oleostearate of sucrose, glucose or methylglucose.

The non-silicone wax(es) that may be used in the cosmetic composition used in step a) are chosen especially from carnauba wax, candelilla wax, esparto grass wax, hydrocarbon waxes including paraffin wax, ozokerite and microcrystalline wax, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina);

other waxes or waxy starting materials that may be used according to the invention are especially marine waxes such as the product sold by the company Sophim under the reference M82, and polyethylene waxes or polyolefin waxes in general.

In a preferred embodiment, the one or more non-silicone-based fatty compounds are selected from oils, waxes, linear or branched alkanes, fatty esters, esters of fatty acids, esters of fatty alcohols, cetyl esters, triglycerides, or a mixture thereof.

The total amount of the one or more non-silicone-based fatty compounds in the compositions, if present, will vary. Nonetheless, in various embodiments, the compositions include about 0.1 to about 20 wt. % of the one or more non-silicone-based fatty compounds, based on the total weight of the compositions. In further embodiments, the compositions include about 0.1 to about 15 wt. %, about 0.1 to about 12 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 20 wt. %, about 1 to about 15 wt. %, about 1 to about 12 wt. %, about 1 to about 10 wt. %, about 1 to about 8 wt. %, about 1 to about 5 wt. %, about 2 to about 20 wt. %, about 2 to about 15 wt. %, about 2 to about 12 wt. %, about 2 to about 10 wt. %, about 2 to about 8 wt. %, about 2 to about 5 wt. %, or about 3 to about 5 wt. %, based on the total weight of the compositions.

### (j) Miscellaneous Ingredients

The compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

### Exclusions

All components that are positively set forth in the instant disclosure may be negatively excluded from the claims, e.g., a claimed composition may be “free,” “essentially free” (or “substantially free”) of one or more components that are positively set forth in the instant disclosure.

In various embodiments, the hair treatment composition is free or essentially free from ethylene carbonate. In further embodiments, the hair treatment composition is free or essentially free from carbonates other than the one or more cyclic carbonates. Similarly, in various embodiments, the composition is free or essentially free from linear carbonates, e.g., dimethyl carbonate, diethyl carbonate, etc. In various embodiments, the composition is free or essentially free from cyclic lactones (e.g., valerolactone, caprolactone, pantolactone, meadowlactone, etc.), free or essentially free from heterocyclic molecules (e.g., 2-oxazolidinone, 2-imidazolidinone, etc.) free or essentially free from sulfones (dimethyl sulfone, 2,3,4,5-tetrahydrothiophene-1,1-dioxide), and/or free or essentially free from ureas (e.g., urea, ethylene urea, etc.).

In yet further embodiments, the hair treatment composition is free or essentially free from carbonates other than propylene carbonate.

In various embodiments, the hair treatment composition is free or essentially free from cationic surfactants. In further embodiments, the hair treatment composition is free or essentially free from cationic conditioning polymers. Similarly, in various embodiments, the composition is free or essentially free from polyquaternium compounds. In further embodiments, the composition is free or essentially free from cationic surfactants and cationic conditioning polymers.

In various embodiments, the hair treatment composition is free or essentially free from anionic surfactants.

In various embodiments, the hair treatment composition is free or essentially free from polymers, copolymers, and crosspolymers formed with acrylate or methacrylate monomers, e.g, free or essentially free from polyacrylic acid and polyacrylate polymers and crosspolymers.

In various embodiments, the hair treatment composition is free or essentially free from N-alkyl-2-mercaptoacetamide. In further embodiments, the hair treatment composition is free or essentially free from all mercaptoacetamides

In various embodiments, the hair treatment composition is free or essentially free from fatty alcohols. In other embodiments, however, the composition includes one or more fatty alcohols, for example, in an amount of about 0.01 to about 10 wt. %, preferably about 0.1 to about 5 wt. %, more preferably in an amount of about 0.5 to about 4 wt. %, based on a total weight of the hair treatment composition. Suitable fatty alcohols, if present, include those having a fatty group with a carbon chain of greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

In various embodiments, the hair treatment composition is free or essentially free from silicones other than the one or more amino-functionalized silicones. Similarly, in various embodiments, the composition is free or essentially free from silicones other than amodimethicone.

In further embodiments, the hair treatment composition is free or essentially free from monosaccharides and disaccharides. For example, the composition is free or essentially free from ribose, arabinose, glucose, fructose, xylose, sucrose, and/or methyl glucoside.

In various embodiments, the hair treatment composition is free or essentially free from formaldehyde, derivatives of formaldehyde, formalin, and compounds that produce formaldehyde upon heating.

In further embodiments, the hair treatment composition is free or essentially free arabinos, keratin, peptides, or a mixture thereof.

Embodiments

In various embodiments, the methods of the instant disclosure are drawn to reducing frizz or increasing smoothness of hair comprising or consisting of:
(i) applying a hair treatment composition to hair in need of reduced frizz or increased smoothness; the composition comprising or consisting of:
   (a) about 1 to about 15 wt. %, preferably about 3 to about 15 wt. %, more preferably about 5 to about 10 wt. % of propylene carbonate, wherein the composition is free from alkylene carbonates, cyclic carbonates other than the propylene carbonate, and N-alkyl-2-mercaptoacetamides;
   (b) about 50 to about 90 wt. %, preferably about 55 to about 85 wt. %, more preferably about 60 to about 80 wt. % of water;
   (c) optionally, about 0.1 to about 10 wt. %, preferably about 1 to about 8 wt. %, more preferably about 1 to about 5 wt. % of one or more silicones, preferably one or more amino-functionalized silicones, for example, one or more amino-functionalized silicones selected from amodimethicone, bis-hydroxy/methoxy amodimethicone, bis-cetearyl amodimethicone, bis (C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof, preferably wherein the one or more amino-functionalized silicones is amodimethicone;
   (d) optionally, one or more film-forming polymers;
   (e) optionally, about 0.1 to about 8 wt. %, preferably about 0.1 to about 6 wt. %, more preferably about 0.5 to about 5 wt. % of one or more polysaccharides, preferably wherein the one or more polysaccharides are selected from starches, modified starches, gums, modified gums, celluloses, modified celluloses, or a mixture thereof, preferably one or more polysaccharides are selected from xanthan gum, gellan gum, sclerotium gum, guar gum and its derivatives, cellulose and its derivatives, and a mixture thereof;
   (f) optionally, about 0.1 to about 5 wt. %, preferably about 0.1 to about 4 wt. %, more preferably about 0.5 to about 3 wt. % of one or more nonionic associative polymeric thickeners, preferably wherein the one or more nonionic associative polymeric thickeners are selected from polyurethane thickeners, preferably selected from polyurethane/polyether thickeners, for example, one or more polyurethane/polyether thickeners selected from PEG-240/HDI copolymer bis-decyltetradeceth-20 ether, PEG-150/stearyl alcohol/SMDI copolymer, PEG-150/decyl alcohol/SMDI copolymer, steareth-100/PEG-136/HDI copolymer, or a mixture thereof;
   (g) optionally, about 0.2 to about 8 wt. %, preferably about 0.5 to about 6 wt. %, more preferably about 1 to about 5 wt. % of one or more surfactants or emulsifiers, preferably one or more nonionic surfactants or emulsifiers, more preferably wherein the one or more nonionic surfactants or emulsifiers are selected from from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl (ether) phosphates, alkylpolyglucosides, and mixtures thereof, for example, one or more alkyl or polyalkyl ethers of poly (ethylene oxide) containing at least one C8-C30 alkyl radical, with a number of ethylene oxide (EO) units ranging from 3 to 200, in particular, selected from laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, or mixtures thereof;
   (h) optionally, about 0.1 to about 15 wt. %, more preferably about 1 to about 12 wt. %, more preferably about 2 to about 10 wt. % of one or more water soluble solvents, preferably wherein the one or more water soluble solvents are selected from glycerin, C1-C6 mono-alcohols, polyols (polyhydric alcohols), glycols, or a mixture thereof, more preferably wherein the one or more water soluble solvents are selected from glycerin, glycols (e.g., ethylene glycol, propylene glycol, butylene glycol, pentylene glycol, caprylyl glycol, etc.), or a combination thereof; and
   (i) optionally, about 0.1 to about 12 wt. %, preferably about 1 to about 10 wt. %, more preferably about 2 to about 8 wt. % of one or more non-silicone-based fatty compounds, preferably wherein the one or more non-silicone-based fatty compounds are selected from oils, waxes, linear or branched alkanes, fatty esters, esters of fatty acids, esters of fatty alcohols, cetyl esters, triglycerides, or a mixture thereof, more preferably wherein the one or more non-silicone-based fatty compounds comprises or consists of one or more linear or branched alkanes, oils, or a mixture thereof;
   (j) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
      wherein all weight percentages are based on a total weight of the composition.
(ii) after rinsing the composition from the hair, subjecting the hair to a heat treatment, wherein the heat treatment heats the hair to a temperature of at least 150° C., preferably wherein the hair is treated with a hot iron, especially a flat iron, at a temperature of about 150 to about 280° C.

The methods of the instant disclosure are particularly useful for treat human hair, in particular human hair of the head (the top of the head as opposed to eyelashes, eyebrows, or facial hair). Treatment with the compositions improves fiber alignment, reduces frizz, and impart smoothness to the hair. The hair is preferably wet or damp when the hair treatment compositions of the instant disclosure are applied, but the hair-treatment composition can also be applied to dry hair. Application of the hair-treatment compositions to the hair will moisten the hair because the compositions are aqueous (contain a large portion of water). Therefore, after massaging or spreading the hair-treatment composition throughout the hair, rinsing the hair-treatment composition from the hair, the hair can be dried, for example, the hair can be dried using a blow dryer. After the hair is dry, the hair can be treated with a thermal treatment (treated with heat). For example, the hair can be treated with a hot iron, in particular, a flat iron. The temperature of the flat iron may be from about 180° C. to about 280° C., or from about 190° C. to about 250° C. Typically, the hot iron is passed over the hair at least once, at least twice, at least three times, or more.

Implementation of the present disclosure is provided by way of the following examples. The examples serve to illustrate the technology without being limiting in nature.

### EXAMPLE 1

| | | | | | | | | | 3x Shampoo + | |
|---|---|---|---|---|---|---|---|---|---|---|
| | | | Initial | | 2 hr @80% | | After 3x | | 2 hr @ 80% | |
| | Composition | | Application | | Humidity | | Shampoo | | Humidity | |
| | A | B | A | B | A | B | A | B | A | B |
| Propylene | 10 | | | | | | | | | |
| carbonate | | | | | | | | | | |
| water | 89 | 100 | | | | | | | | |
| Miscellaneous ¹ | ≤1 | | | | | | | | | |
| pH | 5.7 | 5 | | | | | | | | |
| Smoothness * | | | 4 | 3 | 4 | 2 | 4 | 3 | 3 | 1 |
| Softness * | | | 4 | 3 | 4 | 2 | 4 | 3 | 3 | 1 |
| Alignment * | | | 5 | 3 | 3 | 1 | 4 | 1 | 3 | 1 |
| Frizz control | | | 5 | 3 | 3 | 1 | 4 | 1 | 3 | 1 |

¹ For example, fillers, pH adjusters, preservatives, botanical extracts, fragrances, buffers, stabilizers, vitamins, and/or emollients, etc.
* On a scale of 1-5, 5 is the best, 1 is the worst.
A: Inventive
B: Control

Testing was carried out on curly hair swatches (medium bleached hair with curl pattern 2b) to determine how the compositions in the table above influenced hair. All hair swatches were initially cleansed with a standard shampoo. The shampoo was rinsed from the hair swatches and the hair swatches were towel dried. The damp hair swatches were subsequently treated with one of the compositions set forth in the table above (about 1 gram of composition per gram of hair). After application, all compositions were allowed to remain on the hair for 10 minutes. The compositions were then rinsed from the hair swatches and the hair swatches were blow dried. The dried hair swatches were treated with a hot flat iron at a temperature of about 232° C. The hair was passed through the hot flat iron three times, rinsed with water, and air dried to bring back the curliness. After treatment, the swatches were evaluated for smoothness, softness, alignment, and frizz control on a scale of 1-5 (1 being the worst, and 5 being the best). The swatches were subsequently placed in a humidity chamber at 80% RH and a temperature of 25° C., for two hours. After two hours, the hair swatches were again evaluated for smoothness, softness, alignment, and frizz control on a scale of 1-5. The hair swatches were then cleansed with the same standard shampoo 3 times in a row (repeatedly), air dried, and again evaluated for smoothness, softness, alignment, and frizz control, on a scale of 1-5, and again placed in a humidity chamber at 80% RH and a temperature of 25° C., for two hours. After two hours, the swatches were evaluated for smoothness, softness, alignment, and frizz control on the same scale of 1-5. The results are shown in The Figure. As can be seen, swatches treated according to the inventive treatment showed lasting smoothness, smoothness, softness, and frizz control compared to the control.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated. Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally include one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for reducing frizz or increasing smoothness of hair comprising:
(i) applying a hair treatment composition to hair in need of reduced frizz or increased smoothness; the composition comprising:
(a) about 1 to about 15 wt. % of propylene carbonate; and
(b) water,
wherein the composition is free from alkylene carbonates, cyclic carbonates other than the propylene carbonate, and N-alkyl-2-mercaptoacetamide, and
all weight percentages are based on a total weight of the composition;
(ii) after rinsing the composition from the hair, subjecting the hair to a heat treatment, wherein the heat treatment heats the hair to a temperature of at least 150° C.

2. The method of claim 1, wherein the heat treatment comprises treating the hair with a hot iron at a temperature of about 150 to about 280° C.

3. The method of claim 1, wherein the composition further comprises:
(c) one or more silicones.

4. The method of claim 3, wherein the one or more silicones are selected from amino-functionalized silicones.

5. The method of claim 1, wherein the composition further comprises:
(d) one or more film-forming polymers.

6. The method of claim 5, wherein the one or more film-forming polymers are selected from cationic film-forming polymers, anionic film-forming polymers, nonionic film-forming polymers, amphoteric film-forming polymers, or a combination thereof.

7. The method of claim 1, wherein the composition further comprises:
(e) one or more polysaccharides.

8. The method of claim 7, wherein the one or more polysaccharides are selected from starches, modified starches, gums, modifed gums, celluloses, modified celluloses, or a mixture thereof, preferably one or more polysaccharides are selected from xanthan gum, gellan gum, sclerotium gum, guar gum and its derivatives, cellulose and its derivatives, and a mixture thereof.

9. The method of claim 1, wherein the composition further comprises:
(f) one or more nonionic associative polymeric thickeners.

10. The method of claim 9, wherein the one or more nonionic associative polymeric thickeners are selected from polyurethane thickeners.

11. The method of claim 10, wherein the polyurethane thickeners are polyurethane/polyether thickeners.

12. The method of claim 10, wherein the polyurethane/polyether thickeners are selected from PEG-240/HDI copolymer bis-decyltetradeceth-20 ether, PEG-150/stearyl alcohol/SMDI copolymer, PEG-150/decyl alcohol/SMDI copolymer, steareth-100/PEG-136/HDI copolymer, or a mixture thereof.

13. The method of claim 1, wherein the composition is free from formaldehyde, derivatives of formaldehyde, formalin, and any compounds that produce formaldehyde upon heating.

14. The method of claim 1, wherein the method does not comprise application of an oxidizing compound or an oxidizing composition to the hair.

15. The method of claim 1, wherein the method does not comprise application of thioglycolic acid, thiolactic acid, or salts thereof; and does not comprise application of a peroxide.

16. A method for reducing frizz or increasing smoothness of hair consisting of:
(i) applying a hair treatment composition to hair in need of reduced frizz or increased smoothness; the composition comprising:
(a) about 1 to about 15 wt. % of propylene carbonate; and
(b) water;
(c) optionally, one or more amino-functionalized silicones;
(d) optionally, one or more film-forming polymers;
(e) optionally, one or more polysaccharides;
(f) optionally, one or more nonionic associative polymeric thickeners are selected from polyurethane thickeners;
(g) optionally, one or more surfactants, preferably one or more nonionic surfactant, one or more cationic surfactants, or a combination thereof;
(h) optionally, one or more water soluble solvents;
(i) optionally, one or more non-silicone-based fatty compounds; and
(j) optionally, one or more non-silicone-based fatty compounds;
wherein the composition is free from alkylene carbonates, cyclic carbonates other than the propylene carbonate, and N-alkyl-2-mercaptoacetamides,
all weight percentages are based on a total weight of the composition, and
(ii) after rinsing the composition from the hair, subjecting the hair to a heat treatment, wherein the heat treatment heats the hair to a temperature of at least 150° C.

17. The method of claim 16, wherein the heat treatment comprises drying the hair with a blow dryer followed by treating the hair with a hot iron at a temperature of about 150 to about 280° C.

18. The method of claim 16, wherein the composition is free from formaldehyde, derivatives of formaldehyde, formalin, and any compounds that produce formaldehyde upon heating.

19. The method of claim 1 wherein the method reduces frizz and increases smoothness of the hair to a greater extent than treating the hair with a comparative method that is identical to the method except that the hair treatment composition lacks the propylene carbonate.

20. The method of claim 1 wherein the method reduces frizz and increases smoothness of the hair to a greater extent than treating the hair with a comparative method that is identical to the method except that the hair treatment composition lacks the propylene carbonate.
